(19) United States
(12) Patent Application Publication
(10) Pub. No.: US 2024/0366180 A1
(43) Pub. Date: Nov. 7, 2024

(54) MEDICAL INSTRUMENT NAVIGATION SYSTEM REGISTRATION PROBE WITH DEPTH-FINDING OR IMAGING CAPABILITES

(71) Applicant: Biosense Webster (Israel) Ltd., Yokneam (IL)

(72) Inventors: George Gusein, Acre (IL); Iris Segal, Qiryat Motzkin (IL); Haya Belkind-Shafir, Yokneam (IL); Shmuel Auerbach, Kerem Maharal (IL); Uriel Hod, Haifa (IL)

(21) Appl. No.: 18/594,200

(22) Filed: Mar. 4, 2024

Related U.S. Application Data
(60) Provisional application No. 63/464,267, filed on May 5, 2023.

(51) Int. Cl. A61B 8/08; A61B 5/00; A61B 5/05; A61B 34/20

(52) U.S. Cl. CPC A61B 8/0858; A61B 5/0082; A61B 5/05; A61B 2034/2051

(57) ABSTRACT

An apparatus includes a body, a position sensor, and a depth-finding module or an ultrasound imager. The position sensor is fixedly positioned relative to a distal end of the body. The position sensor is configured to generate a signal indicating a real-time position of the position sensor within three-dimensional space. The depth-finding module or an ultrasound imager is fixedly positioned relative to the position sensor. The distal end of the body is configured to emit energy from the depth-finding module or ultrasound imager.

### PRIORITY

This application claims priority to U.S. Provisional Pat. App. No. 63/464,267, entitled “Medical Instrument Navigation System Registration Probe with Depth-Finding or Imaging Capabilities,” filed May 5, 2023, the disclosure of which is incorporated by reference herein, in its entirety.

### BACKGROUND

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. An example of an electromagnetic IGS navigation system that may be used in IGS procedures is the CARTO® 3 System by Biosense-Webster, Inc., of Irvine, California. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, some instruments can include sensors (e.g., electromagnetic coils that emit electromagnetic fields and/or are responsive to externally generated electromagnetic fields), which can be used to perform the procedure while the sensors send data to the computer indicating the current position of each sensor-equipped instrument. The computer correlates the data it receives from the sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. The surgeon is thus able to know the precise position of each sensor-equipped instrument by viewing the video monitor even if the surgeon is unable to directly visualize the instrument itself at its current location within the body.

One function that may be performed by an IGS system is obtaining one or more reference points that may be used to correlate various preoperatively obtained images with a patient's actual position during a procedure. This act may be referred to as patient registration. Such registration may be performed by using a positionally tracked instrument (e.g., a registration probe whose tip position may be detected in three-dimensional space) to trace or touch one or more positions on a patient's face. At each touch point, the IGS system may register that point in three-dimensional space; and, using a number of registered points, determine the position of the affected area in three-dimensional space. Once the affected area is fully mapped or registered, it may be correlated with preoperative images in order to provide a seamless IGS experience across varying types of preoperative images during the performance of the procedure.

Some patient registration devices may be placed in contact with the skin of the patient. However, due swelling of tissue, depression of the registration device into the skin, or other structural deformations, it is possible that a skin-contacting patient registration device might not always provide accurate and reliable registration data on certain anatomical structures of the patient. While several systems and methods have been made and used in connection with IGS navigation systems, it is believed that no one prior to the inventors has made or used the invention described in the appended claims.

### BRIEF DESCRIPTION OF THE DRAWINGS

The drawings and detailed description that follow are intended to be merely illustrative and are not intended to limit the scope of the invention as contemplated by the inventors.

FIG. 1 depicts a schematic view of an example of a surgery navigation system being used on a patient seated in an example of a medical procedure chair;

FIG. 2 depicts a front perspective view of an example of a registration probe having a position sensing tip;

FIG. 3 depicts a front elevation view of a patient during a registration procedure using the registration probe of FIG. 2;

FIG. 4 depicts a cross-sectional schematic view of an example of a registration probe contacting an outer surface of a head of a patient, with no deformation of soft tissue on the head of the patient;

FIG. 5 depicts a cross-sectional schematic view of the registration probe of FIG. 4 contacting an outer surface of a head of a patient, where the soft tissue on the head of the patient is deformed in the contact region;

FIG. 6 depicts a cross-sectional schematic view of another example of a registration probe contacting an outer surface of a head of a patient, where the soft tissue on the head of the patient is deformed in the contact region;

FIG. 7 depicts a cross-sectional schematic view of another example of a registration probe contacting an outer surface of a head of a patient, where the soft tissue on the head of the patient is deformed in the contact region; and

FIG. 8 depicts a schematic view of registration points in a real-time ultrasonic image being correlated with registration points in a preoperative image.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

### I. Example of an Image Guided Surgery Navigation System

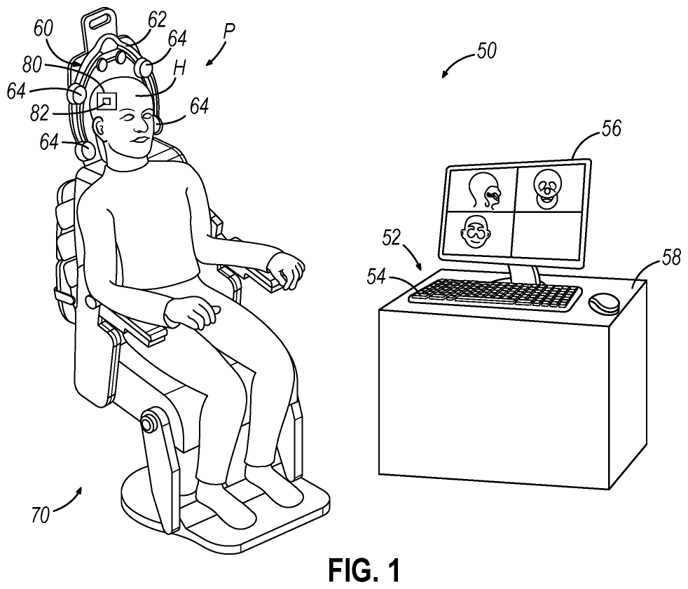

When performing a medical procedure within a head of a patient (P), it may be desirable to have information regarding the position of an instrument within the head (H) of the patient (P), particularly when the instrument is in a location where it is difficult or impossible to obtain an endoscopic view of a working element of the instrument within the head of the patient (P). FIG. 1 shows an example of an IGS navigation system (50) enabling a medical procedure to be performed within a head (H) of a patient (P) using image guidance. In addition to or in lieu of having the components and operability described herein IGS navigation system (50) may be constructed and operable in accordance with at least some of the teachings of U.S. Pat. No. 7,720,521, entitled “Methods and Devices for Performing Procedures within the Ear, Nose, Throat and Paranasal Sinuses,” issued May 18, 2010, the disclosure of which is incorporated by reference herein, in its entirety; and/or U.S. Pat. No. 10,561,370, entitled “Apparatus to Secure Field Generating Device to Chair,” issued Feb. 18, 2020, the disclosure of which is incorporated by reference herein, in its entirety.

IGS navigation system (50) of the present example comprises a field generator assembly (60), which comprises a set of magnetic field generators (64) that are integrated into a horseshoe-shaped frame (62). Field generators (64) are operable to generate alternating magnetic fields of different frequencies around the head (H) of the patient (P). An instrument may be inserted into the head (H) of the patient (P). Such an instrument may include one or more position sensors as described in greater detail below. In the present example, frame (62) is mounted to a chair (70), with the patient (P) being seated in the chair (70) such that frame (62) is located adjacent to the head (H) of the patient (P). By way of example only, chair (70) and/or field generator assembly (60) may be configured and operable in accordance with at least some of the teachings of U.S. Pat. No. 10,561,370, entitled “Apparatus to Secure Field Generating Device to Chair,” Issued Feb. 18, 2020, the disclosure of which is incorporated by reference herein, in its entirety. In some other variations, the patient (P) lies on a table; and field generator assembly (60) is positioned on or near the table.

IGS navigation system (50) of the present example further comprises a processor (52), which controls field generators (64) and other elements of IGS navigation system (50). For instance, processor (52) is operable to drive field generators (64) to generate alternating electromagnetic fields; and process signals from the instrument to determine the location of a navigation sensor or position sensor in the instrument within the head (H) of the patient (P). Processor (52) comprises a processing unit (e.g., a set of electronic circuits arranged to evaluate and execute software instructions using combinational logic circuitry or other similar circuitry) communicating with one or more memories. Processor (52) of the present example is mounted in a console (58), which comprises operating controls (54) that include a keypad and/or a pointing device such as a mouse or trackball. A physician uses operating controls (54) to interact with processor (52) while performing the surgical procedure.

While not shown, the instrument that is used with IGS navigation system (50) may include a navigation sensor or position sensor that is responsive to positioning within the alternating magnetic fields generated by field generators (64). A coupling unit (not shown) may be secured to the proximal end of the instrument and may be configured to provide communication of data and other signals between console (58) and the instrument. The coupling unit may provide wired or wireless communication of data and other signals.

In some versions, the navigation sensor or position sensor of the instrument may comprise at least one coil at or near the distal end of the instrument. When such a coil is positioned within an alternating electromagnetic field generated by field generators (64), the alternating magnetic field may generate electrical current in the coil, and this electrical current may be communicated along the electrical conduit(s) in the instrument and further to processor (52) via the coupling unit. This phenomenon may enable IGS navigation system (50) to determine the location of the distal end of the instrument within a three-dimensional space (i.e., within the head (H) of the patient (P), etc.). To accomplish this, processor (52) executes an algorithm to calculate location coordinates of the distal end of the instrument from the position related signals of the coil(s) in the instrument. Thus, a navigation sensor may serve as a position sensor by generating signals indicating the real-time position of the sensor within three-dimensional space.

Processor (52) uses software stored in a memory of processor (52) to calibrate and operate IGS navigation system (50). Such operation includes driving field generators (64), processing data from the instrument, processing data from operating controls (54), and driving display screen (56). In some implementations, operation may also include monitoring and enforcement of one or more safety features or functions of IGS navigation system (50). Processor (52) is further operable to provide video in real time via display screen (56), showing the position of the distal end of the instrument in relation to a video camera image of the patient's head (H), a CT scan image of the patient's head (H), and/or a computer-generated three-dimensional model of the anatomy within and adjacent to the patient's nasal cavity. Display screen (56) may display such images simultaneously and/or superimposed on each other during the surgical procedure. Such displayed images may also include graphical representations of instruments that are inserted in the patient's head (H), such that the operator may view the virtual rendering of the instrument at its actual location in real time. By way of example only, display screen (56) may provide images in accordance with at least some of the teachings of U.S. Pat. No. 10,463,242, entitled “Guidewire Navigation for Sinuplasty,” issued Nov. 5, 2019, the disclosure of which is incorporated by reference herein, in its entirety. In the event that the operator is also using an endoscope, the endoscopic image may also be provided on display screen (56). The images provided through display screen (56) may help guide the operator in maneuvering and otherwise manipulating instruments within the patient's head (H).

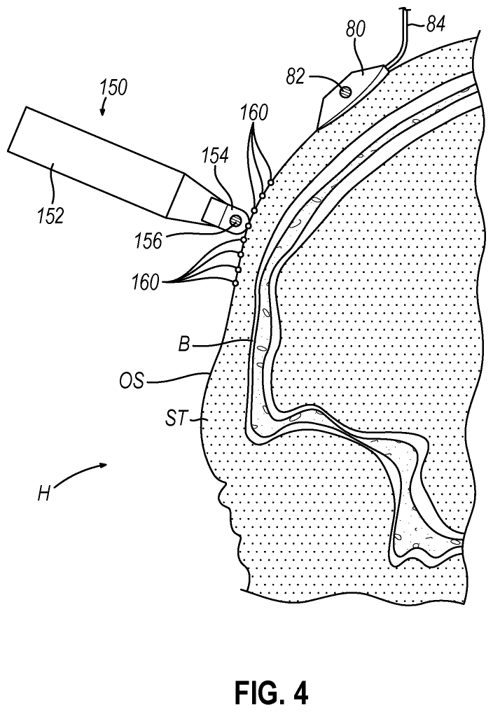

In the present example, field generators (64) are in fixed positions relative to the head (H) of the patient (P), such that the frame of reference for IGS navigation system (50) (i.e., the electromagnetic field generated by field generators (64)) does not move with the head (H) of the patient (P). In some instances, the head (H) of the patient (P) may not remain completely stationary relative to field generators (64) throughout the duration of a medical procedure, such that it may be desirable to track movement of the head (H) of the patient (P) during a medical procedure. To that end, a patient tracking assembly (80) is secured to the head (H) of the patient (P) in this example. Patient tracking assembly (80) may be secured to the head (H) via an adhesive, via one or more screws, or in any other suitable fashion. Patient tracking assembly (80) includes a position sensor (82), which is in communication with processor (52), such as via a cable (84) (see FIGS. 4-7). In some versions, position sensor (82) is wirelessly coupled with processor (52), such that cable (84) is omitted.

Position sensor (82) is configured to generate signals indicating the real-time position of position sensor (82) in response to an alternating electromagnetic field generated by field generators (64). By way of example only, position sensor (82) may comprise one or more coils. The signals generated by position sensor (82) are communicated to processor (52), such that processor (52) may process signals from position sensor (82) to determine the real-time position of position sensor (82) in three-dimensional space. With patient tracking assembly (80) being firmly secured to the head (H) of the patient (P), patient tracking assembly (80) may move unitarily with the head (H) of the patient (P). Accordingly, signals from position sensor (82) may effectively indicate the real-time position of the head (H) of the patient in three-dimensional space.

After patient tracking assembly (80) is secured to the head (H) of the patient (P), an operator may insert one or more position sensor equipped medical instruments (e.g., ENT shaver, suction cannula, balloon dilation catheter, electrosurgical instrument, etc.) into the head (H) of the patient (P). Signals from position sensors of such instruments may be communicated to processor (52), thereby enabling processor (52) to determine the real-time positions of such instruments in three-dimensional space. With processor (52) knowing the real-time position of the head (H) of the patient (P) in three-dimensional space based on signals from position sensor (82), and with processor (52) knowing the real-time position of a medical instrument in three-dimensional space based on signals from one or more position sensors in the medical instrument, processor (52) may accurately determine the real-time position of the medical instrument in the head (H) of the patient (P). Processor (52) may thereby drive display screen (56) to display an indicator (e.g., crosshairs, etc.) showing the real-time position of the medical instrument in the head (H) of the patient (P) as described above. By way of example only, processor (52) may drive display screen (56) to display an indicator (e.g., crosshairs, etc.) to show the real-time position of the medical instrument in the head (H) of the patient (P) as an overlay on one or more images of at least a portion of the head (H) of the patient (P).

### II. Example of Patient Registration with IGS System

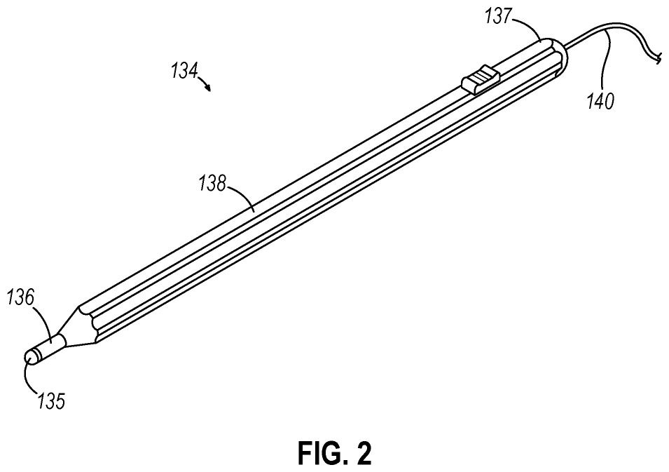

As noted above it may be necessary to register a patient (P) with an IGS navigation system (50) in order to allow processor (52) to initially correlate a real-time position of the patient (P) with one or more preoperative images (e.g., CT images, MRI images, three-dimensional model, etc.) of the patient (P), to thereby allow processor (52) to track and visually indicate the real-time position of a position sensor-equipped instrument in the patient (P) via display screen (56). As also noted above, such a registration process may be carried out using a registration probe. FIG. 2 shows an example of a registration probe (134). Registration probe (134) of this example comprises a rigid elongate body (138) having a distal end (136) and a proximal end (137). Distal end (136) terminates in a rounded distal tip (135).

A guidewire (140) may be inserted into rigid elongate body (138) so that an end of guidewire (140) rests against the interior of rounded distal tip (135). The distal end of guidewire (140) includes a position sensor (e.g., one or more coils as described above) that is spaced at a known distance from distal tip (135). Thus, distal tip (135) may be used to touch a registration point on the face of the patient (P), which will place the position sensor of guidewire (140) at a known distance from the registration point on the face of the patient (P) that is being contacted by distal tip (135). In this configuration, instrument (134) may be used to perform the registration and calibration process associated with IGS navigation system (50) by touching rounded distal tip (135) to each registration point while providing another input to the system, such as interacting with a foot pedal or button, speaking a voice command, or another similar input, to cause the registration touch to be recorded. In some versions, registration probe (134) includes a contact sensor (not shown) that senses when distal tip (135) contacts the face of the patient (P).

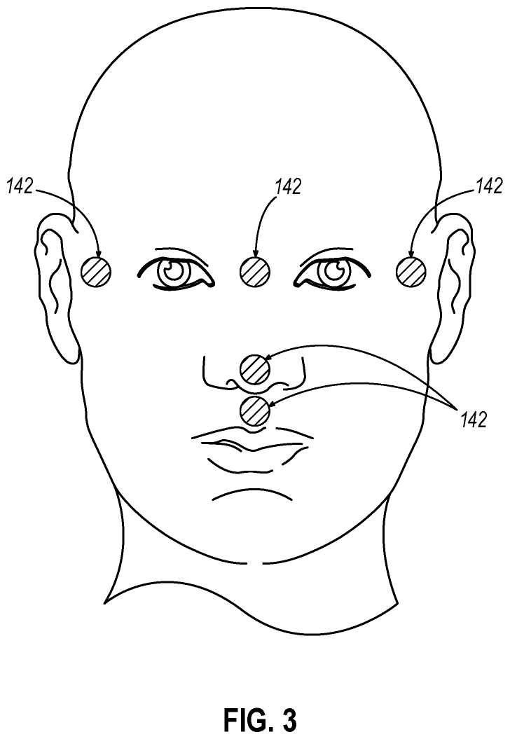

FIG. 3 shows a front elevation view of a patient showing an example of a patient registration procedure using registration probe (134). The view of FIG. 3 might be rendered on display screen (56) of IGS navigation system (50) during a registration procedure to guide the operator; and may show one or more registration points (142) that must be calibrated or registered using registration probe (134). In the example of FIG. 3, this registration process may require five touches of registration probe (134) at different registration points (142) on the face of the patient (P) to be completed. In other scenarios, the number of registration points (142) may be more or fewer than five. By way of further example only, registration points (142) may be shown on display screen (56) in one color before they are registered with a touch of the registration probe (134); and may change to a different color or otherwise indicate calibration after a touch of registration probe (134). In some versions, one or more lasers are used to project registration points (142) on the face of the patient (P), such that the operator must engage the face of the patient with rounded distal tip (135) at each point illuminated by the laser(s).

In addition to the foregoing, registration probe (134) may be configured and operable in accordance with at least some of the teachings of U.S. Pat. No. 10,779,891, entitled “System and Method for Navigation of Surgical Instruments,” issued Sep. 22, 2020, the disclosure of which is incorporated by reference herein, in its entirety.

FIG. 4 shows another example of a registration probe (150) in use. Registration probe (150) of this example may be configured and operable like registration probe (134), except for any differences described below. Registration probe (150) of this example includes a body (152) having a distal tip (154) with a position sensor (156) integrated into distal tip (154). Position sensor (156) may comprise one or more coils. The signals generated by position sensor (156) are communicated to processor (52), such that processor (52) may process signals from position sensor (156) to determine the real-time position of position sensor (156) in three-dimensional space.

Body (152) is sized and configured to be grasped by the hand of an operator. Registration probe (150) is positioned such that distal tip (154) softly contacts an outer surface (OS) of soft tissue (ST) (e.g., skin, etc.) over bone (B) in the head (H) of the patient (P). In particular, distal tip (154) is moved to contact several registration points (160) along the outer surface (OS) of the head of the patient (P). Position sensor (156) captures position data at each registration point (160) to thereby register the patient with one or more preoperative images stored in IGS navigation system (50) as described above. In the example shown in FIG. 4, the soft tissue (ST) between the bone (B) and the region of the outer surface (OS) that is being contacted by distal tip (154) is not deformed. Thus, the distance between each registration point (160) and the underlying bone (B) is consistent with the same distance as is captured in the one or more preoperative images stored in IGS navigation system (50). This consistency provides an accurate registration process.

### III. Examples of Registration Probes with Features to Account for Soft Tissue Deformation

As noted above, an accurate registration process may be dependent, in part, on consistency between the distance from each registration point (160) and the underlying bone (B) with the same distance as is captured in the one or more preoperative images stored in IGS navigation system (50). In cases where the distance from each registration point (160) and the underlying bone (B) is not consistent with the same distance as is captured in the one or more preoperative images stored in IGS navigation system (50), then the registration process may be inaccurate. Such deformation may occur under various circumstances. For instance, the soft tissue (ST) within the registration region may be swollen due to a medication taken by the patient (P) between the time the preoperative images were captured and the time the registration is performed. As another scenario, the soft tissue (ST) within the registration region may be swollen due to a wound sustained by the patient (P) between the time the preoperative images were captured and the time the registration is performed. As yet another scenario, the elasticity of the skin within the registration region may have changed between the time the preoperative images were captured and the time the registration is performed. The deformation of soft tissue (ST) within the registration region may also be inadvertently caused by the operator, such as by the operator pressing distal tip (154) against the head (H) of the patient (P) with enough force to cause deformation of the soft tissue (ST).

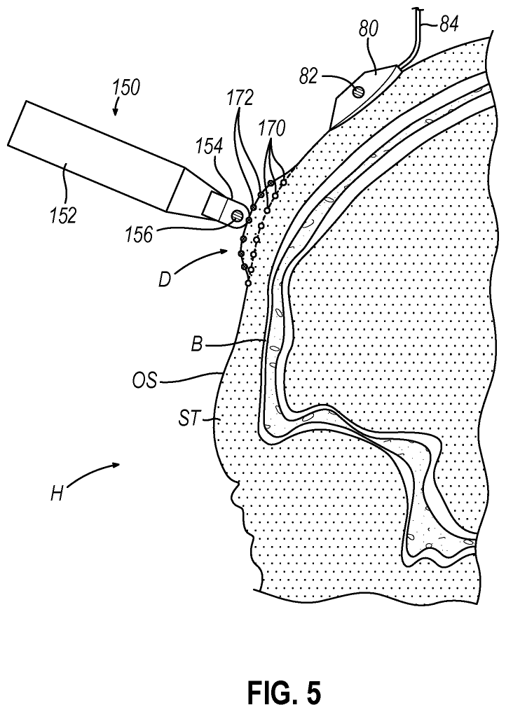

FIG. 5 shows an example of a scenario where the soft tissue (ST) has a deformation (D) in the form of a swell in the registration region. The principles described below may nevertheless also apply to other forms of deformation, including but not limited to depressions. It should be understood that, in this example, deformation (D) was not present at the time the preoperative images were captured; but is present during the registration process. As shown in FIG. 5, distal tip (154) contacts the outer surface (OS) of the head (H) of the patient (P) at registration points (172) that are spaced further away from the underlying bone (B) than the corresponding registration points (170) that would be contacted in the absence of the deformation (D). In other words, registration points (170) correspond to positions along the outer surface (OS) in the preoperative images that were captured before deformation (D) was formed. When completing the registration process, processor (52) would thus “expect” registration points (172) to be at the positions of registration points (170). The offset of the actual registration points (172) from the expected registration points (170) will impart inaccuracy to the registration process. This may in turn result in inaccuracies in subsequent renderings by IGS system (50) of the real-time position of position sensor-equipped medical instruments that are disposed in the head (H) of the patient (P).

In view of the foregoing, it may be desirable to provide a variation of registration probe (134, 150) that is configured to account for any deformation (D) in soft tissue (ST) over bone (B); or is otherwise not sensitive to any deformation (D) in soft tissue (ST) over bone (B). Examples of such alternative registration probes are described in greater detail below.

### A. Example of Registration Probe with Depth-Finding Ultrasound

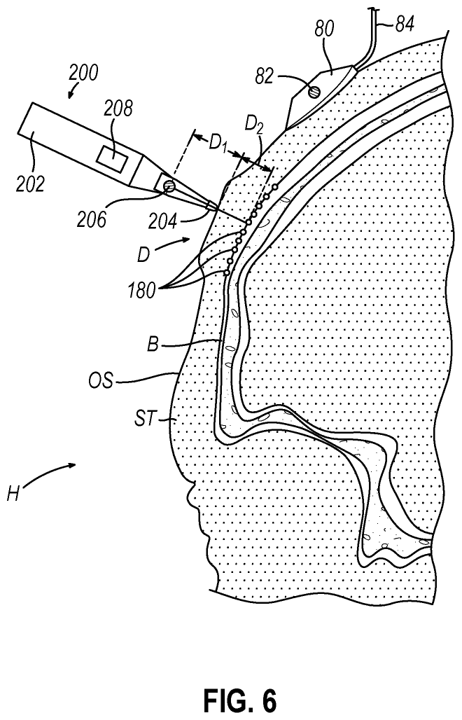

FIG. 6 shows a registration probe (200) that includes a body (202) having a distal tip (204), with a position sensor (206) fixed at a known distance (D1) from distal tip (204). Position sensor (206) may comprise one or more coils. The signals generated by position sensor (206) are communicated to processor (52), such that processor (52) may process signals from position sensor (206) to determine the real-time position of position sensor (206) in three-dimensional space. Body (202) is sized and configured to be grasped by the hand of an operator. Unlike registration probes (134, 150) described above, registration probe (200) of this example further includes an ultrasonic depth-finding module (208). Ultrasonic depth-funding module (208) may include any suitable components, such as a transducer containing piezoelectric elements, an acoustic waveguide, and/or other features. While ultrasonic depth-finding module (208) is shown as being positioned within body (202), it should be understood that depth-finding module (208) may instead be positioned at any other suitable location in registration probe (200), including but not limited to being embedded in distal tip (204).

Ultrasonic depth-finding module (208) may be placed in communication with processor (52) just like position sensor (206), such as via wire or wirelessly, such that ultrasonic depth-finding module (208) may communicate data to processor (52) as described in greater detail below. Ultrasonic depth-finding module (208) is configured to drive distal tip (204) to emit ultrasonic waves through soft tissue (ST), with such ultrasonic waves being reflected by the bone (B) back to distal tip (204). Ultrasonic depth-finding module (208) may further process these reflected ultrasonic waves in order to determine a distance (D2) from the outer surface (OS) to the bone (B). Since the distance (D1) between position sensor (206) and the outer surface (OS) is already known when distal tip (204) is in contact with the outer surface (OS), and since the distance (D2) from the outer surface (OS) to the bone (B) is determined through the ultrasonic depth-finding capabilities of ultrasonic depth-finding module (208), processor (52) may readily determine the distance between position sensor (206) and the bone (B) (e.g., by adding the distances (D1, D2) together).

To the extent that any deformation (D) in the soft tissue (ST) causes any changes in the distance (D2) between the outer surface (OS) and the bone (B) along the registration region, such changes in that distance (D2) may be readily accounted for based on the soft tissue (ST) depth data acquired via depth-finding module (208) in real time. It should also be understood that the use of registration probe (200) may allow the operator to capture registration points (180) on the bone (B) in the head (H) of the patient (P). By having the registration points (180) on the bone (B) instead of being on the soft tissue (ST), it may be easier for processor (52) to correlate the registration points (180) captured via registration probe (200) with corresponding points in the preoperative images since the bone (B) may be far less susceptible to deformation during the period between the capture of the preoperative images and the registration process. In other words, registration points (180) on bone (B) may tend to provide greater consistency throughout the period between the capture of the preoperative images and the registration process; as compared to registration points (160, 170, 172) on the outer surface (OS) of soft tissue (ST). Thus, the process of using registration probe (200) may tend to be more reliable, and provide more consistently accurate patient registrations, than the process of using registration probes (134, 150).

In the example provided above, depth-finding module (208) employs ultrasonic waves to determine the distance (D2) between the outer surface (OS) of soft tissue (ST) and bone (B) (i.e., the thickness of the soft tissue (ST) over bone (B)). However, in other variations, depth-finding module (208) may utilize other modalities to determine the distance (D2) between the outer surface (OS) of soft tissue (ST) and bone (B). For instance, any suitable form of a time-of-flight (ToF) sensor may be used to provide a variation of depth-finding module (208), including but not limited to ToF sensors that employ light such as infrared light or high frequency laser light. As another merely illustrative example, depth-finding module (208) may provide an ultra-wideband radar, in addition to or in lieu of providing ultrasonic emissions, to determine the distance (D2) between the outer surface (OS) of soft tissue (ST) and bone (B). As another merely illustrative example, depth-finding module (208) may provide an light detection and ranging (LiDAR), in addition to or in lieu of providing ultrasonic emissions, to determine the distance (D2) between the outer surface (OS) of soft tissue (ST) and bone (B). Alternatively, depth-finding module (208) may provide emissions elsewhere within the electromagnetic radiation spectrum, including but not limited to within the spectrum of light or within the spectrum of radio waves.

### B. Example of Registration Probe with Ultrasonic Imaging

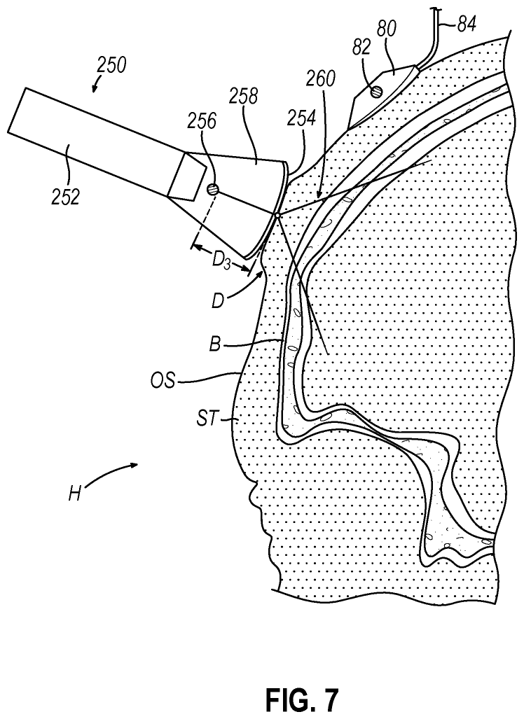

As noted above, registration probe (200) employs the use of ultrasonic waves for depth-finding purposes; but not for capturing ultrasonic images. However, some variations of registration probe (200) may in fact provide the capability of capturing ultrasonic images. Moreover, such captured ultrasonic images may be utilized to provide registration of the patient with one or more preoperative images via IGS navigation system (50). To that end, FIG. 7 shows an example of a registration probe (250) that includes a body (252) having a distal end (254), with a position sensor (256) fixed at a known distance (D3) from distal end (254). Position sensor (256) may comprise one or more coils. The signals generated by position sensor (256) are communicated to processor (52), such that processor (52) may process signals from position sensor (256) to determine the real-time position of position sensor (256) in three-dimensional space. Body (252) is sized and configured to be grasped by the hand of an operator. Unlike registration probes (134, 150) described above, registration probe (250) of this example further includes an ultrasonic imaging head (258). Ultrasonic imaging head (258) may include any suitable components, such as a transducer containing piezoelectric elements, an acoustic waveguide, and/or other features.

Ultrasonic imaging head (258) may be placed in communication with processor (52) just like position sensor (256), such as via wire or wirelessly, such that ultrasonic imaging head (258) may communicate data to processor (52) as described in greater detail below. Ultrasonic imaging head (258) is configured to emit an ultrasonic imaging wavefield (260) from distal end (254). This ultrasonic imaging wavefield (260) passes through the soft tissue (ST) and reaches bone (B), such that ultrasonic imaging head (258) may generate ultrasonic images of the regions of bone (B) within ultrasonic imaging wavefield (260). In some cases, these ultrasonic images may be used to determine the distance between distal end (254) and bone (B), similar to the depth-finding capabilities of ultrasonic depth-finding module (208) described above. Since the distance (D3) between position sensor (256) and the outer surface (OS) is already known when distal end (256) is in contact with the outer surface (OS), and since the distance from the outer surface (OS) to the bone (B) may be determined through the ultrasonic images captured by ultrasonic imaging head (258), processor (52) may readily determine the distance between position sensor (256) and the bone (B). This may allow registration probe (250) to capture registration points on the bone (B), similar to the capture of registration points (180) as described above in the context of registration probe (200).

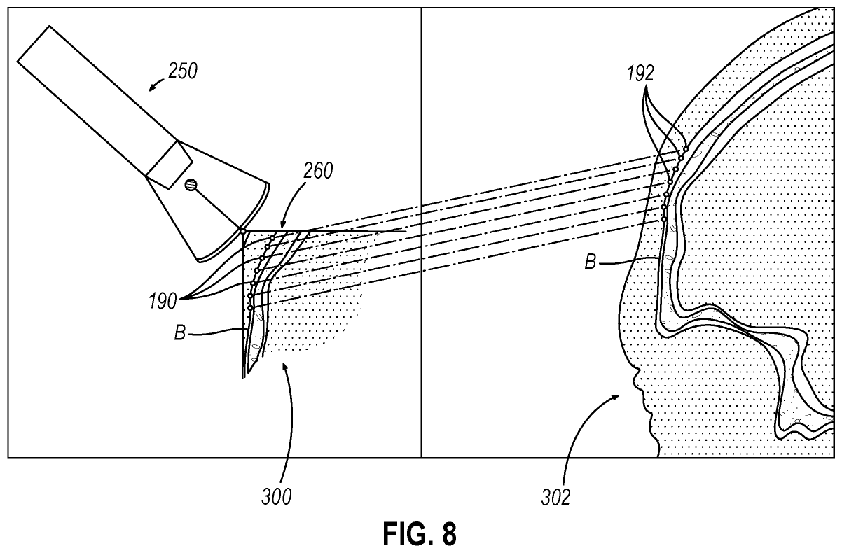

In another example of use of registration probe (250), processor (52) may analyze ultrasonic images captured by ultrasonic imaging head (258) to identify certain structures of the bone (B) using image recognition algorithms; and correlate those identified structures of the bone (B) with corresponding structures in the preoperative images. For instance, FIG. 8 shows an example of an ultrasonic image (300) in which processor (52) has automatically identified several registration points (190) through processing of ultrasonic images captured by ultrasonic imaging head (258). FIG. 8 also shows an example of a preoperative image (302) (e.g., a CT scan image, MRI scan image, etc.), where processor (52) has automatically mapped points (192) corresponding to the same anatomical structures of bone (B) associated with registration points (190) in ultrasonic image (300). It should therefore be understood that processor (52) may rely on image processing techniques, utilizing real-time ultrasonic images captured by ultrasonic imaging head (258) of registration probe (250), in addition to relying on real-time position data from position sensor (256), to achieve registration of the patient (P) with preoperative images in IGS navigation system (50).

### IV. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: (a) a body having a distal end; (b) a position sensor, the position sensor being fixedly positioned relative to the distal end, the position sensor being configured to generate a signal indicating a real-time position of the position sensor within three-dimensional space; and (c) an ultrasonic assembly, the ultrasonic assembly being fixedly positioned relative to the position sensor, the ultrasonic assembly comprising a transducer operable to generate ultrasonic waves, the distal end being configured to emit the ultrasonic waves generated by the transducer.

### Example 2

The apparatus of Example 1, the ultrasonic assembly being configured to determine a depth of soft tissue between bone in a patient and an outer surface of the patient in contact with the distal end.

### Example 3

The apparatus of Example 2, further comprising a processor configured to determine a distance between the position sensor and the bone in the patient based at least in part on a combination of a known distance between the position sensor and the distal end and the depth of soft tissue determined by the ultrasonic assembly.

### Example 4

The apparatus of Example 3, further comprising an image guided surgery system external to the body, the processor being contained in the image guided surgery system.

### Example 5

The apparatus of Example 4, the image guided surgery system further comprising a field generator assembly, the field generator assembly being operable to generate an electromagnetic field, the position sensor being configured to generate a signal indicating a real-time position of the position sensor within three-dimensional space in response to the electromagnetic field.

### Example 6

The apparatus of any of Examples 3 through 5, the processor being further configured to register a patient with one or more preoperative images based at least in part on the determined distance between the position sensor and the bone in the patient and the signal indicating a real-time position of the position sensor within three-dimensional space.

### Example 7

The apparatus of any of Examples 1 through 6, the ultrasonic assembly being configured to generate ultrasonic images.

### Example 8

The apparatus of Example 7, further comprising a processor configured to register a patient with one or more preoperative images based at least in part on one or more ultrasonic images generated by the ultrasonic assembly and the signal indicating a real-time position of the position sensor within three-dimensional space.

### Example 9

The apparatus of Example 8, the processor being further configured to identify structural features of bone in the one or more ultrasonic images generated by the ultrasonic assembly through one or more image processing algorithms.

### Example 10

The apparatus of Example 9, the processor being further configured to correlate the structural features of bone identified from the one or more ultrasonic images with corresponding structural features of bone in one or more preoperative images.

### Example 11

The apparatus of Example 10, the processor being further configured to map a plurality of registration points from bone in the one or more ultrasonic images with corresponding points in the one or more preoperative images.

### Example 12

An apparatus, comprising: (a) a body having a distal end; (b) a position sensor, the position sensor being fixedly positioned relative to the distal end by a first distance, the position sensor being configured to generate a signal indicating a real-time position of the position sensor within three-dimensional space; and (c) a depth-finding module, the depth-finding module being fixedly positioned relative to the position sensor, the depth-finding module being operable to determine a second distance representing a real-time depth of soft tissue between bone in a patient and a contact point between an outer surface of the patient and the distal end.

### Example 13

The apparatus of Example 12, further comprising a processor, the processor being configured to determine a real-time distance from the position sensor to the bone in the patient underlying the contact point based on a combination of the first and second distances.

### Example 14

The apparatus of Example 13, the processor being further configured to register a patient with one or more preoperative images based at least in part on the determined real-time distance and the signal indicating a real-time position of the position sensor within three-dimensional space.

### Example 15

The apparatus of any of Examples 12 through 14, the depth finding module being operable to: (i) emit ultrasonic energy through tissue between the outer surface of the patient and the bone in the patient, and (ii) receive ultrasonic energy reflected by the bone in the patient to thereby determine the second distance.

### Example 16

The apparatus of any of Examples 12 through 15, the depth finding module being operable to: (i) emit light through tissue between the outer surface of the patient and the bone in the patient, and (ii) receive light reflected by the bone in the patient to thereby determine the second distance.

### Example 17

The apparatus of Example 16, the emitted light being within the infrared spectrum.

### Example 18

The apparatus of any of Examples 16 through 17, the emitted light comprising laser light.

### Example 19

The apparatus of any of Examples 12 through 18, the depth finding module being operable to determine the second distance via ultra-wideband radar.

### Example 20

An apparatus, comprising: (a) a body having a distal end; (b) a position sensor, the position sensor being fixedly positioned relative to the distal end by a first distance, the position sensor being configured to generate a signal indicating a real-time position of the position sensor within three-dimensional space; and (c) an ultrasonic imaging head, the ultrasonic imaging head module being fixedly positioned relative to the position sensor, the ultrasonic imaging head being operable to generate ultrasonic images of bone underlying a region of contact between the distal end and an outer surface of the patient.

### Example 21

The apparatus of Example 20, further comprising a processor configured to register a patient with one or more preoperative images based at least in part on one or more ultrasonic images generated by the ultrasonic assembly and the signal indicating a real-time position of the position sensor within three-dimensional space.

### Example 22

The apparatus of Example 21, the processor being further configured to identify structural features of bone in the one or more ultrasonic images generated by the ultrasonic assembly through one or more image processing algorithms.

### Example 23

The apparatus of Example 22, the processor being further configured to correlate the structural features of bone identified from the one or more ultrasonic images with corresponding structural features of bone in one or more preoperative images.

### Example 24

The apparatus of Example 23, the processor being further configured to map a plurality of registration points from bone in the one or more ultrasonic images with corresponding points in the one or more preoperative images.

### Example 25

A method, comprising: (a) positioning a registration probe against an outer surface of a patient at a first contact point; (b) capturing position data via a position sensor of the registration probe while the registration probe is positioned against the outer surface of the patient at the first contact point, the position data indicating a real-time position of the position sensor within three-dimensional space; (c) emitting energy through soft tissue underlying the outer surface of the patient, the energy being reflected by bone under the soft tissue; and (d) capturing data from the reflected energy, the captured data representing one or both of: (i) a depth of soft tissue between the first contact point and the bone, or (ii) an image of the bone.

### Example 26

The method of Example 25, further comprising: (a) positioning the registration probe against an outer surface of a patient at a second contact point; and (b) repeating steps (b) through (d) while the registration probe is at the second contact point.

### Example 27

The method of any of Examples 25 through 26, further comprising generating a registration point at least in part on the position data indicating a real-time position of the position sensor within three-dimensional space and the data captured from the reflected ultrasonic waves.

### Example 28

The method of Example 27, further comprising registering the patient with one or more preoperative images based at least in part on the generated registration point.

### Example 29

The method of any of Examples 27 through 28, the registration point being positioned on the bone.

### Example 30

The method of any of Examples 25 through 29, the emitted energy comprising ultrasonic energy.

### Example 31

The method of any of Examples 25 through 30, the emitted energy comprising electromagnetic radiation.

### Example 32

The method of Example 31, the electromagnetic radiation having a wavelength within the spectrum of light.

### Example 33

The method of Example 31, the electromagnetic radiation having a wavelength within the spectrum of radio waves.

### Example 34

The method of Example 33, the electromagnetic radiation comprising ultra-wideband radiation.

### Example 35

A method, comprising: (a) receiving position data from a position sensor of a registration probe, the position data indicating a real-time position of the position sensor within three-dimensional space; (b) receiving data from an ultrasonic assembly of the registration probe, the data from the ultrasonic assembly representing one or both of: (i) a depth of soft tissue between a point of patient contact between a distal end of the registration probe and bone underlying the point of patient contact, or (ii) an image of the bone underlying the point of patient contact; and (c) registering the patient with one or more preoperative images based at least in part on a combination of the position data and the data from the ultrasonic assembly.

### Example 36

A method, comprising: (a) receiving position data from a position sensor of a registration probe, the position data indicating a real-time position of the position sensor within three-dimensional space; (b) receiving data from a depth-finding assembly of the registration probe, the data from the depth-finding assembly representing a depth of soft tissue between a point of patient contact between a distal end of the registration probe and bone underlying the point of patient contact; and (c) registering the patient with one or more preoperative images based at least in part on a combination of the position data and the data from the depth-finding assembly.

### V. Miscellaneous

It should be understood that any of the teachings, expressions, embodiments, examples, etc. described herein may be combined with any of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one skilled in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus, comprising:
(a) a body having a distal end;
(b) a position sensor, the position sensor being fixedly positioned relative to the distal end, the position sensor being configured to generate a signal indicating a real-time position of the position sensor within three-dimensional space; and
(c) an ultrasonic assembly, the ultrasonic assembly being fixedly positioned relative to the position sensor, the ultrasonic assembly comprising a transducer operable to generate ultrasonic waves, the distal end being configured to emit the ultrasonic waves generated by the transducer.

2. The apparatus of claim 1, the ultrasonic assembly being configured to determine a depth of soft tissue between bone in a patient and an outer surface of the patient in contact with the distal end.

3. The apparatus of claim 2, further comprising a processor configured to determine a distance between the position sensor and the bone in the patient based at least in part on a combination of a known distance between the position sensor and the distal end and the depth of soft tissue determined by the ultrasonic assembly.

4. The apparatus of claim 3, further comprising an image guided surgery system external to the body, the processor being contained in the image guided surgery system.

5. The apparatus of claim 4, the image guided surgery system further comprising a field generator assembly, the field generator assembly being operable to generate an electromagnetic field, the position sensor being configured to generate a signal indicating a real-time position of the position sensor within three-dimensional space in response to the electromagnetic field.

6. The apparatus of claim 3, the processor being further configured to register a patient with one or more preoperative images based at least in part on the determined distance between the position sensor and the bone in the patient and the signal indicating a real-time position of the position sensor within three-dimensional space.

7. The apparatus of claim 1, the ultrasonic assembly being configured to generate ultrasonic images.

8. The apparatus of claim 7, further comprising a processor configured to register a patient with one or more preoperative images based at least in part on one or more ultrasonic images generated by the ultrasonic assembly and the signal indicating a real-time position of the position sensor within three-dimensional space.

9. The apparatus of claim 8, the processor being further configured to identify structural features of bone in the one or more ultrasonic images generated by the ultrasonic assembly through one or more image processing algorithms.

10. The apparatus of claim 9, the processor being further configured to correlate the structural features of bone identified from the one or more ultrasonic images with corresponding structural features of bone in one or more preoperative images.

11. The apparatus of claim 10, the processor being further configured to map a plurality of registration points from bone in the one or more ultrasonic images with corresponding points in the one or more preoperative images.

12. An apparatus, comprising:
(a) a body having a distal end;
(b) a position sensor, the position sensor being fixedly positioned relative to the distal end by a first distance, the position sensor being configured to generate a signal indicating a real-time position of the position sensor within three-dimensional space; and
(c) a depth-finding module, the depth-finding module being fixedly positioned relative to the position sensor, the depth-finding module being operable to determine a second distance representing a real-time depth of soft tissue between bone in a patient and a contact point between an outer surface of the patient and the distal end.

13. The apparatus of claim 12, further comprising a processor, the processor being configured to determine a real-time distance from the position sensor to the bone in the patient underlying the contact point based on a combination of the first and second distances.

14. The apparatus of claim 13, the processor being further configured to register a patient with one or more preoperative images based at least in part on the determined real-time distance and the signal indicating a real-time position of the position sensor within three-dimensional space.

15. The apparatus of claim 12, the depth finding module being operable to:
(i) emit ultrasonic energy through tissue between the outer surface of the patient and the bone in the patient, and
(ii) receive ultrasonic energy reflected by the bone in the patient to thereby determine the second distance.

16. The apparatus of claim 12, the depth finding module being operable to:
(i) emit light through tissue between the outer surface of the patient and the bone in the patient, and
(ii) receive light reflected by the bone in the patient to thereby determine the second distance.

17. The apparatus of claim 16, the emitted light being within the infrared spectrum.

18. The apparatus of claim 16, the emitted light comprising laser light.

19. The apparatus of claim 12, the depth finding module being operable to determine the second distance via ultra-wideband radar.

20. A method, comprising:
(a) positioning a registration probe against an outer surface of a patient at a first contact point;
(b) capturing position data via a position sensor of the registration probe while the registration probe is positioned against the outer surface of the patient at the first contact point, the position data indicating a real-time position of the position sensor within three-dimensional space;
(c) emitting energy through soft tissue underlying the outer surface of the patient, the emitted energy being reflected by bone under the soft tissue; and
(d) capturing data from the reflected energy, the captured data representing one or both of:
(i) a depth of soft tissue between the first contact point and the bone, or
(ii) an image of the bone.
